(19) United States
(12) Patent Application Publication
(10) Pub. No.: US 2026/0207104 A1
(43) Pub. Date: Jul. 23, 2026

(54) APPARATUS FOR MONITORING PHYSIOLOGICAL SIGANLS WITH DETACHABLE ELECTRODE PATCH UNIT

(71) Applicant: BO PI, SAN DIEGO, CA (US)

(72) Inventors: BO PI, SAN DIEGO, CA (US); IRENE PI, SAN DIEGO, CA (US)

(21) Appl. No.: 19/031,664

(22) Filed: Jan. 18, 2025

(51) Int. Cl. A61B 5/259; A61B 5/266; A61B 5/282

(52) U.S. Cl. CPC A61B 5/259; A61B 5/266; A61B 5/282

(57) ABSTRACT

An apparatus for monitoring physiological signals is provided in the present application. The apparatus includes a physiological main unit and an electrode patch unit. The physiological main unit includes a sensor assembly, and the electrode patch unit is detachably engaged to the physiological main unit; the electrode patch unit includes a plurality of conductive electrodes being affixed to a human body to detect physiological signals from the human body; the sensor assembly is electrically coupled to the conductive electrodes of the electrode patch unit and configured for collecting the physiological signals from the electrode patch unit The apparatus for monitoring physiological signals as provided in the present application has a better wear experience and improvement of physiological signal collection.

### TECHNICAL FIELD

This present application generally relates to an apparatus for monitoring physiological signals including heart signals such as an electrocardiogram (ECG) data, which in particular has a detachable electrode patch unit.

### BACKGROUND

Abnormal heart signals such as arrhythmias may cause various types of symptoms, for example, loss of consciousness, palpitations, dizziness, or even death. These symptoms are often an indicator of significant underlying heart disease; it is therefore important for people to discover abnormal heart signals timely to address such heart disease symptoms.

Heart signal monitoring may be typically accomplished by use of an ECG monitoring device, such as a Holter monitor or other similar smart device, which can be designed to be affixed to a chest of a user and worn for at least a few days. The ECG monitoring device can collect and record some cardiac rhythm parameters including ECG data and one or more other physiological parameters continuously, and these parameters can further be available for processing and analysis to identify the aforesaid symptoms. As such, the user can take necessary treatment in time, such as pacemaker implantation or percutaneous catheter ablation, which can successfully ameliorate these problems and prevent significant symptoms.

The existing wearable ECG monitoring apparatus generally has an unduly big form and is inconvenience for a person to wear, and once being wore may impedes the normal activities of daily living. Moreover, if the ECG monitoring apparatus is not properly affixed to the chest, it may be unable to collect the accurate ECG data.

It is, therefore, desired to provide a new physiological monitoring apparatus, which is smaller and convenience for proper wearing, to improve the wear experience and be possible to provide a better diagnostic yield.

### SUMMARY

The examples of implementations described in the present application provide an apparatus for monitoring physiological signals with a better wearing experience and improvement of physiological signal collection.

In an implementation according to one aspect of the present application, an apparatus for monitoring physiological signals is provided. The apparatus includes a physiological main unit with a sensor assembly, and an electrode patch unit being detachably engaged to the physiological main unit; the electrode patch unit includes a plurality of conductive electrodes being affixed to a human body to detect physiological signals from the human body; the sensor assembly is electrically coupled to the conductive electrodes of the electrode patch unit and configured for collecting the physiological signals from the electrode patch unit.

In an implementation according to another aspect of the present application, a charge dock for providing charging and data transmission for a physiological main unit is disclosed, the physiological main unit includes a sensor assembly for collecting physiological signals from a human body, the charge dock includes a dock housing being detachably engaged to the physiological main unit; a rear cover being fixed to the dock housing to form a receiving space; and a circuit board module received in the receiving space and being electrically connected to the physiological main unit; the circuit board module includes a printed circuit board and a cable connector arranged at the printed circuit board, the cable connector is configured for being connected to a cable for providing a charge current to the physiological main unit and transmitting the physiological signals collected by the physiological main unit to a host.

The above and other aspects and features are described in greater detail in the following detailed description taken in conjunction with the accompanying drawings and claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 illustrates a schematic diagram of an example of an ECG monitor apparatus according to an exemplary embodiment of the present application, the ECG monitor apparatus including an ECG main unit and an electrode patch unit.

FIG. 2 illustrates an exploded diagram of an exemplary ECG main unit of the ECG monitor apparatus according to an embodiment of the present application.

FIG. 3 illustrates a schematic view of a rear case of the ECG main unit of FIG. 2.

FIG. 4 illustrates a top, assembled view of the ECG main unit of FIG. 2.

FIG. 5 illustrates a side view of the ECG main unit of FIG. 2.

FIG. 6 illustrates a bottom view of the ECG main unit of FIG. 2.

FIG. 7 illustrates a schematic view of an exemplary electrode patch unit of the ECG monitor apparatus according to an embodiment of the present application.

FIG. 8 illustrates an exploded diagram of the exemplary electrode patch unit of FIG. 7.

FIG. 9 illustrates a schematic view of a bracket of the exemplary electrode patch unit of FIG. 7.

FIG. 10 illustrates a top, assembled view of the electrode patch unit of FIG. 8.

FIG. 11 illustrates a side view of the electrode patch unit of FIG. 8.

FIG. 12 illustrates an exemplary assembly process of the ECG main unit and the electrode patch unit according to an embodiment of the present application.

FIG. 13 illustrates a schematic diagram of an exemplary charge dock applicable to the ECG main unit according to an exemplary embodiment of the present application.

FIG. 14 illustrates an exploded diagram of the charge dock of FIG. 13.

FIG. 15 illustrates a top, assembled view of the charge dock of FIG. 13.

FIG. 16 illustrates a side view of the charge dock of FIG. 13.

FIG. 17 illustrates an exemplary electrical communication between the ECG main unit and the charge dock according to an embodiment of the present application.

### DETAILED DESCRIPTION

Detail technical solutions in various embodiments of the present application will be described hereinafter in conjunction with the attached drawings.

The following description is directed to a number of various embodiments. The described embodiments, however, may be implemented and/or varied in many different ways. For example, the described embodiments may be implemented in a long-term wearable physiological monitoring apparatus for use of physiological signal monitoring or health condition diagnosis, which has a small form and can be worn conveniently and comfortably for one or more week or even longer. Such physiological monitoring apparatus may be a patch-based ECG monitoring apparatus or Holter monitor including a number of features as described in the following description to provide a better wear experience and improvement of physiological signal collection, which can facilitate and/or enhance the patient experience and to make diagnosis of heath condition including cardiac arrhythmias to be more accurate.

Referring to FIG. 1, a schematic diagram of an exemplary physiological monitoring apparatus 10 is shown according an embodiment of the present application. In the present embodiment the physiological monitoring apparatus 10 is taken as an ECG monitoring apparatus 10 as in example. The ECG monitoring apparatus 10 includes an ECG main unit 100 and an electrode patch unit 200; the electrode patch unit 200 is detachably engaged to the ECG main unit 100, and the electrode patch unit 200 can be a disposable member which can be discarded and replaced by a new one after use.

The electrode patch unit 200 can be affixed onto a human body, for example, onto a left chest position near the heart of the human body; the electrode patch unit 200 may use electrode leads or traces, which are conformal contact with the human body, to detect physiological electrical signals including cardiac rhythm parameters such as ECG data from the human body. The ECG main unit 100 can be engaged or assembled to the electrode patch unit 200 via a mechanical engagement configuration therebetween, and moreover, the ECG main unit 100 is also electrically connected to the electrode leads or traces of the electrode patch unit 200, so as to collect and record the physiological signals from the electrode patch unit 200. The physiological signals may further be amplified and processed by the ECG main unit 100 to obtain some available heart signals, e.g., cardiac rhythm signals including ECG data, these signals can be used for available diagnosis cardiac rhythm or other heart health condition.

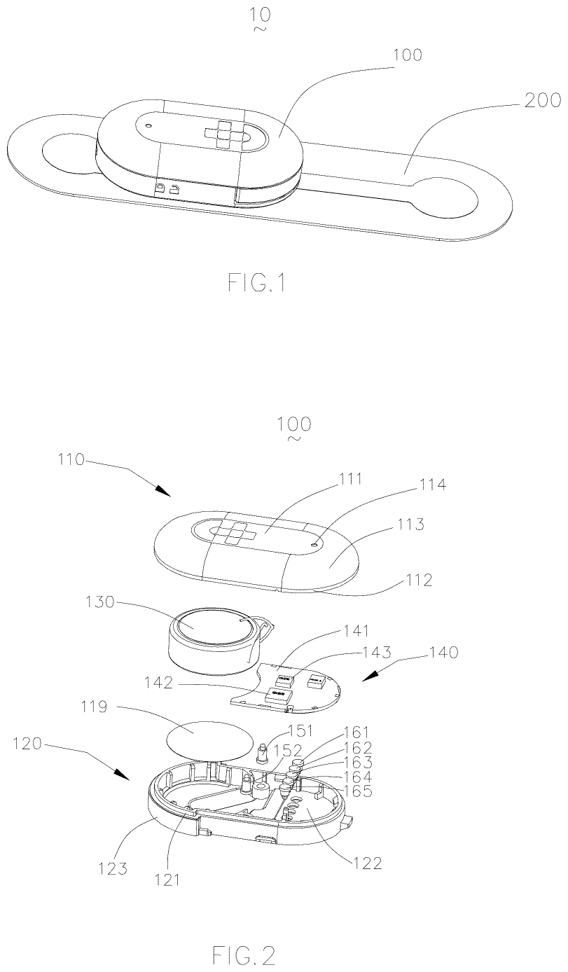

Referring also to FIGS. 2-6, the ECG main unit 100 includes a top cover 110, a rear case 120, a battery 130, an ECG sensor assembly 140, a pair of charging pins 151 and 152, and a plurality of data pins 161~165. The top cover 110 and the rear case 120 cooperates to each other to form a housing with an accommodating space; the battery 130 and the ECG sensor assembly 140 is received in the accommodating space. The battery 130 is configured to provide a power voltage to the ECG sensor assembly 140. The pair of charging pins 151 and 152 and the plurality of data pins 161~165 are electrically connected to the ECG main unit 100, and are arranged to be exposed on a bottom of the rear case 120, such that each of the data pins 161~165 can electrically contact with a corresponding terminal or pad in the electrode patch unit 200 when the ECG main unit 100 is engaged to the electrode patch unit 200.

Although various shapes or configurations may be adapted, in the illustrated embodiment the housing which is formed by the top cover 110 and the rear case 120 has an approximate oval or pebble shape to facilitate a wearing experience. The top cover 110 and the rear case 120 may be made of engineering plastics such as Polycarbonate and Acrylonitrile Butadiene Styrene (PC+ABS) to reduce total size and weight of the ECG main unit 100.

In one embodiment, the top cover 110 may include a top planar part 111, a lower edge 112 for being fixed to a top edge 121 of the rear case 120, and a cambered part 113 arranged between the top planer part 111 and the lower edge 112. The top planar part 111, the cambered part 113 and the lower edge 112 may be integrated into a one-piece structure. In addition, an indicator window 114 may be disposed in the top planar part 111, which is located in a position corresponding to an indicator LED mounted on the ECG sensor assembly 140, and thus light emitted by the indicator LED can be transmitted through the indicator window 114 and make a user be aware of an operation state of the ECG main unit 100.

The rear case 120 includes a top edge 121, a bottom plate 122, and an enclosure wall 123 arranged between the top 121 and the bottom plate 122, which can also be integrated into a one-piece structure. The bottom plate 122 may have an approximate oval shape; the enclosure wall 123 extends upwards from a peripheral margin of the bottom plate 122, and has an approximate oval ring-shaped structure. The top edge 121 is arranged on a top end of the enclosure wall 123, and is used for being engaged to the low edge 112 of the top cover 110 to form the accommodated space.

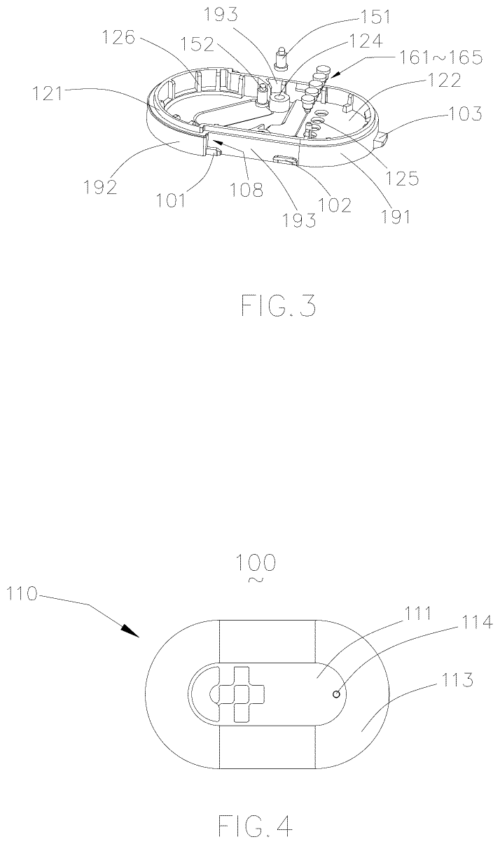

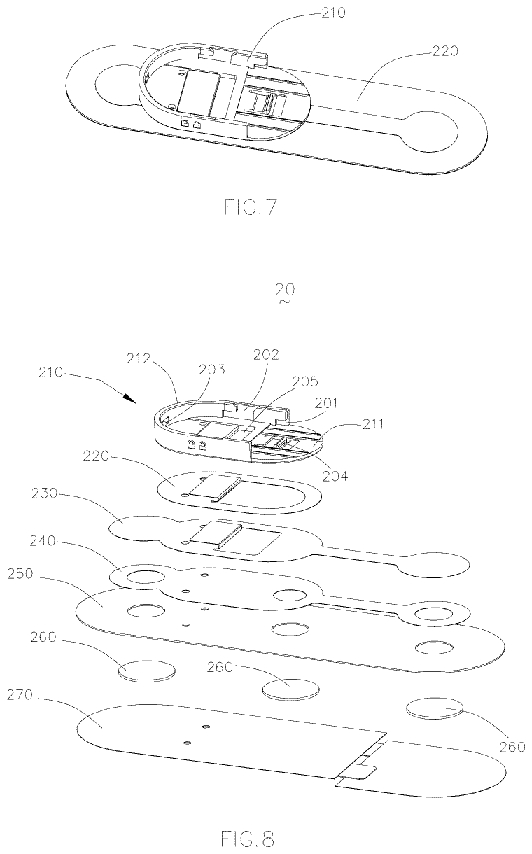

Referring also to FIG. 3, the enclosure wall 123 may include a pair of arc-walls 191 and 192 opposite to each other, and a pair of plane-walls 193 and 194 being connected between the pair of first arc-wall 191 and 192. The pair of first arc-wall 191 and 192 may be arranged at a front end and a back end respectively, which serve as a connecting wall 191 and a free wall 192. A profile of the connecting wall 191 is in accordance with that of the top cover 110 in order to ensure a surface evenness of the ECG main unit 100 when the top cover 110 is assembled to the rear case 120, and a radius of the connecting wall 191 is slightly less than the free wall 192. The connecting wall 191 includes a connecting protrusion 103 being arranged at an outer middle portion thereof, the connecting protrusion 103 is adapted for fitting into a receiving groove 203 (as shown in FIG. 8) in the electrode patch unit 200. In particular, a plurality of reinforce ribs 124 may be formed and distributed on the inner surface of the enclosure wall 123, and be connected to the bottom plate 122.

The pair of plane-walls 193 and 194 are parallel to each other, and are smoothly extended from two ends of the connecting wall 191 respectively; moreover, distal ends of the pair of the plane-walls 193 and 194 are joints to the free wall 192 respectively. Since the radius of the free wall 192 is slightly greater than the connecting wall 191, a recess 108 is formed at a joint area between each of the plane-walls 193 and 194 and a corresponding end of the free wall 192.

In addition, each of the plane-walls 193 and 194 further includes a stopper block 101 and a sliding block 102 which extending from a lower edge thereof. The sliding block 102 is arranged adjacent to the connecting wall 191, and the stopper block 101 is arranged adjacent to the free wall 192; for example, the stopper blocker 101 may be located at a bottom corner of the recess 108. An extending height of the stopper block 101 and the sliding block 102 from the plan-wall 193 and 194 is less than a depth of the recess 108, such that the stopper block 101 and the sliding block 102 would not extend beyond the recess 108 to ensure the ECG main unit to be compact.

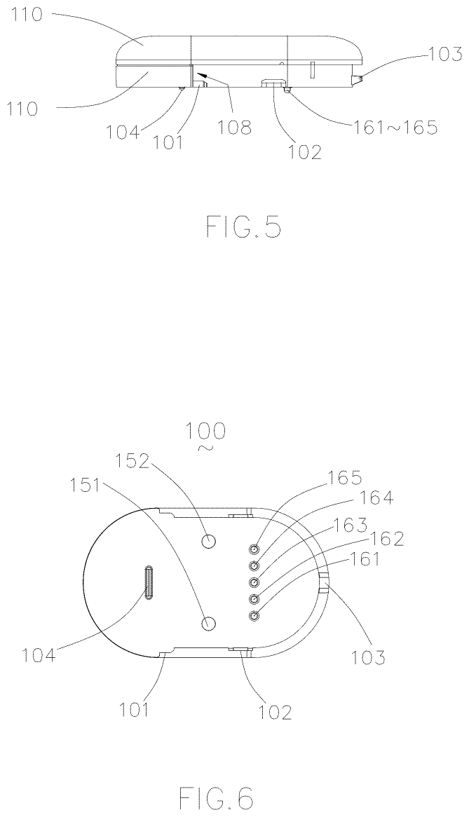

Furthermore, as shown in FIG. 6, the bottom plate 122 may include a locking protrusion 104 extending downwards from a lower surface thereof, and being located substantially adjacent to the free wall 192 under the battery 130. The locking protrusion 104 is adapted for fitting to a latch member 204 in the electrode patch unit 200 (as shown in FIG. 8); and optionally, the locking protrusion 104 may be clipped by the latch member 204 to constitute a locking mechanism, which can make the ECG main unit 100 be engaged and assembled to electrode patch unit 200 more tightly.

The battery 130 may be a chargeable button cell such as a lithium battery, and can be attached onto an upper surface of the bottom plate 122 via an adhesive member 119, the adhesive member 119 may for example be a double-sided tape in a round shape. The battery 130 can be located near the free wall 192. The ECG sensor assembly 140 is arranged near the connecting wall 191 and is electrically connected to the battery 130 for receiving the power voltage provided by the battery 130.

The ECG sensor assembly 140 may include a main carrier board 141, an ECG sensor chip 142, and a controller 143. The main carrier board 141 may for example be a printed circuit board; the ECG sensor chip 142, the controller 143 and some other circuit elements are electrically mounted on the main carrier board 141. The controller 143 may be adapted to control an operation of the ECG main unit 100, and the ECG sensor chip 142 is adapted to collect and record the physiological signals from the electrode patch unit 200, and to process these physiological signals obtain some available heart signals, e.g., cardiac rhythm signal, including ECG data. In addition, the ECG sensor assembly 140 may further include a wireless communicating unit such as a WIFI unit or a Bluetooth unit, which can be wirelessly connected to a host to transmitting the ECG data or other physiological signals to the host for further diagnosis such as heart condition or cardiac arrhythmias diagnosis.

Both of the pair of charging pins 151 and 152 and the plurality of data pins 161~165 may use pogo pins. A typical pogo pin may include a barrel and an elastic conductive probe elastically received in the barrel. An end of the barrel (namely, a barrel contact end) and the elastic conductive probe serve as two connecting ends of the pogo pins. For installation of the charging pins 151 and 152, a pair of mounting posts 124 may be formed and stand upright from the bottom plate 122; each of the mounting posts 124 has a countersunk through hole for receiving a respective one of the charging pins 151 and 152. For example, each of the charging pins 151 and 152 may be inserted from a lower surface of the bottom plate 122 and pass through the countersunk though hole in the mounting post 124, with an elastic probe thereof penetrating out of the mounting posts 124 for electrically contacting a corresponding pad on the main carrier board 141, and with a barrel contact end thereof being exposed on the bottom of the rear case 120 for electrically coupled to a charge dock for receiving a charge current.

In addition, the bottom plate 122 may further include a plurality of mounting structures 125 similar to the mounting posts 124 for receiving the data pins 161~165; in the illustrated embodiment however, the data pins 161~165 is disposed in a reverse manner in relative to the power pins 151 and 152. In other words, the data pins 161~165 can be installed from the upper surface of the bottom plate 122 and pass through the mounting structures 125, such that an elastic probe of each of the data pins 161~165 penetrates out of the bottom of the rear case 120 for being electrically coupled to the electrode patch unit 200, and a barrel contact end of each of the data pins 161~165 being exposed on the upper surface of the bottom plate 122 for being electrically coupled to the ECG sensor assembly 140.

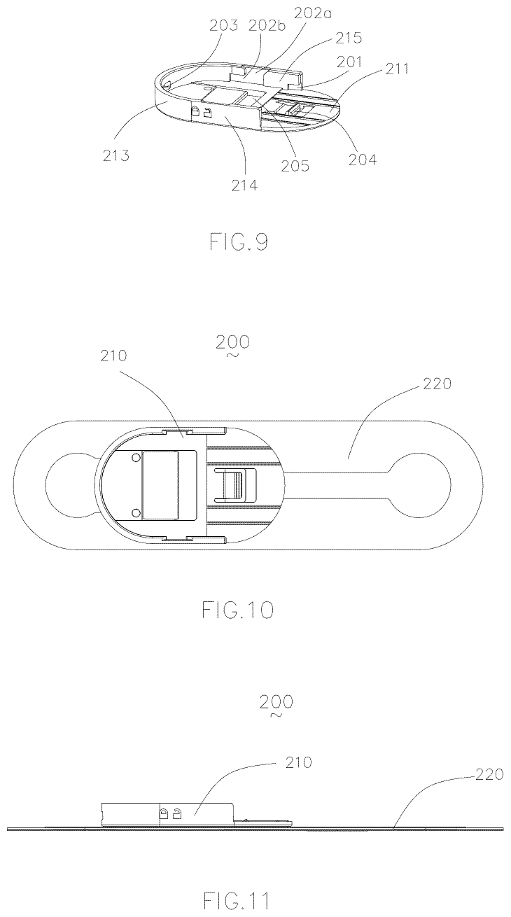

Referring to FIGS. 7-11, the electrode patch unit 200 is a low-cost disposable unit, which includes a bracket 210, a line film 230, a medical tape 250, a plurality of conductive electrodes 260, and a release film 270. The bracket 210 has a profile and configuration in accordance with the ECG main unit 100, to enable the ECG main unit 100 to be engaged to the electrode patch unit 200. Specifically, as illustrated in FIG. 8, the bracket 210 may include a base plate 211 and a semi-enclosure wall 212 extending from a peripheral margin of the base plate 211. Referring also to FIG. 9, the semi-enclosure wall 212 includes an arc-wall 213 corresponding to the connecting wall 191 of the rear case 120, and a pair of plane-walls 214 and 215 parallel to each other and corresponding to the plane-walls 193 and 194 respectively. In one embodiment, the bracket 210 may also be made of engineering plastics such as Polycarbonate and Acrylonitrile Butadiene Styrene (PC+ABS).

A receiving groove 203 is formed on an inner surface of the arc-wall 213, which is configured for receiving the connecting protrusion 103 when the ECG main unit 100 is engaged or assembled to the electrode patch unit 200. Two ends of the arc-wall 213 are connected to the pair of plane-walls 214 and 215 respectively. Each of the plane-walls 214 and 215 includes a sliding groove 202 and a receiving notch 201 formed on a bottom portion of an inner surface thereof. The receiving notch 201 is arranged at a distal end of the plane-walls 214 and 215, which is adapted for receiving and limiting a position of the stopper block 101 of the rear case 120.

The sliding groove 202 is arranged adjacent to the arc-wall 213, and includes a guiding portion 202a and a sliding portion 202b. The guiding portion 202a and the sliding portion 202b are communicated with each other to form an L-shaped groove. The guiding portion 202a is formed above the sliding portion 202b, and is configured for guiding a corresponding sliding block 102 of the plane-wall 193 or 194 to move into the sliding portion 202 b. The sliding portion 202b is adapted for enabling the corresponding sliding block 102 to slide therein towards the arc-wall 213.

Moreover, the base plate 211 of the bracket 210 includes a latch member 204 as mentioned above. The latch member 204 is formed on an upper surface of the base plate 211 and at a region corresponding to the locking protrusion 104 of the rear case 120, and is adapted for latching on the locking protrusion 104 after the main ECG unit 100 is engaged or assembled to the electrode patch unit 200. Additionally, the base plate 211 further includes an opening 205 formed at a region corresponding to the data pins 161~165, and with this configuration each of the elastic probes of the data pins 161~165 can be electrically coupled to the line film 230.

The line film 230 includes a plurality of conductive traces therein, and can be attached to a bottom of the base plate 211 via an adhesive member 220 such as a double-sided tape. The line film 230 may further be attached to the medical tape 250 via another adhesive member 240, which may also be a double-sided tape.

The conductive electrodes 260 can be formed by conductive gels as illustrated in FIG. 8. The conductive electrodes 260 are located on a lower surface of the medical tape 250, and is further electrically connected to the electrical traces in the line film 230. In this embodiments, three conductive gels are provided only for illustration, and it should be noted that in other embodiments, the number of the conductive electrodes 260 can be extended to more; for example, five, seven or even twelve conductive electrodes can alternatively be provided to detect the physiological signals from a human body. Each of the conductive electrodes 260 may be a round-shaped electrode having a sufficient size in order to improve a signal collection effect. To ensure the electrical connection between the line film 230 and the conductive electrodes 260, both of the medical tape 250 and the adhesive member 240 includes openings at the positions of the conductive electrodes 260.

The release film 270 is arranged at a bottom of the electrode patch unit 200, and covers the conductive electrodes 260 to protect the conductive electrodes 260 before use. When the ECG monitoring apparatus 10 is used, the release film 270 can be removed from the electrode patch unit 200, and the electrode patch unit 200 can then be affixed onto a human body (for example, onto a left chest of the human body) via the medical tape 250, and accordingly, the conductive electrodes 260 can detect the physiological signals from the human body. To facilitate a user to remove the release film 270 more convenient, the release film 270 can be divided into two release wings, as illustrated in FIG. 8.

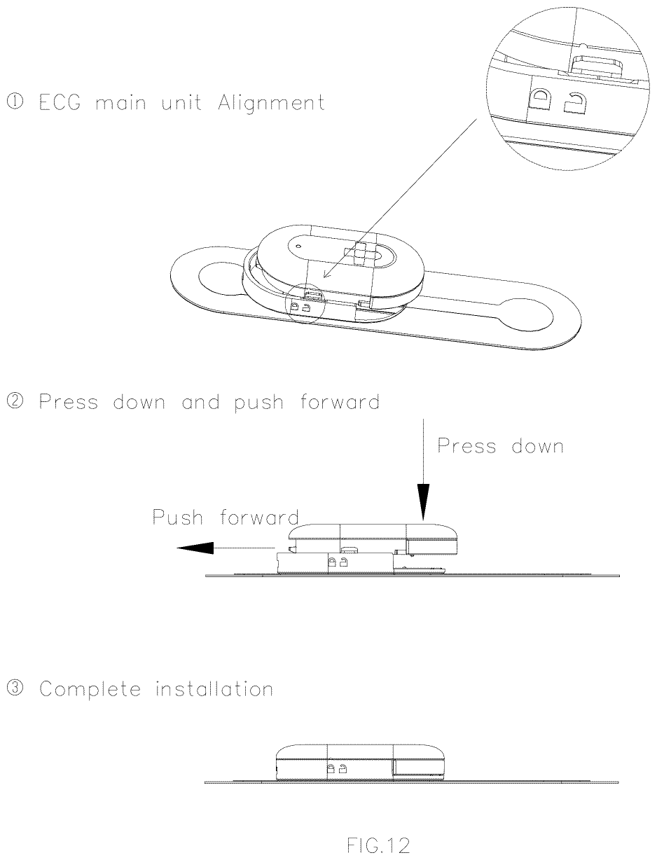

Referring to FIG. 12, an exemplary assembly process of the ECG main unit 100 and the electrode patch unit 200 according to an embodiment of the present application is shown. In assembly, first of all, the ECG main unit 100 may be moved by a user towards the electrode patch unit 200, with each sliding block 102 of the rear case 120 being aligned with a corresponding sliding groove 202 of the bracket 210. Secondly, the user presses the ECG main unit 100 down to make the sliding block 102 passes down through the guiding portion 202a of the sliding groove 202 and reach the sliding portion 202b, and then, the ECG main unit 100 can be pushed forward to enable the sliding block 102 to slide along the sliding portion 202b, until the connecting protrusion 103 of the rear case 120 is received in the receiving groove 203 of the bracket 210, and the stopper block 101 of the rear case 120 is received and limited in the receiving notch 201; at the same time, the locking protrusion 104 of the rear case 120 is latched on by the latch member 204 of the bracket 210. As such, the ECG main unit 100 can be engaged or assembled to the electrode patch unit 200 tightly to constitute the physiological monitoring apparatus 10. Finally, the physiological monitoring apparatus 10 can be worn by the user by affixing the electrode patch unit 200 to the human body as described above. As can be seen, a user can easily and conveniently wear the physiological monitoring apparatus 10 onto his body when it is needed to continuously monitor the physiological signals including cardiac rhythm parameters such as ECG data.

Moreover, in the illustrated embodiment, the connecting protrusion 103 and the receiving groove 203, the sliding block 102 and the sliding groove 202, the stopper block 101 and the receiving notch 201, as well as the locking protrusion 104 and the latching member 121b, cooperate together to form a stable mechanical engagement configuration for the ECG main unit 100 and the electrode patch unit 200. With this configuration, a reliability of physiological signal monitoring can be ensured, and besides, the physiological monitoring apparatus 10 can be in a waterproof form, thus the user can keep wearing the physiological monitoring apparatus 10 even taking exercise, swimming or taking a bath or a shower.

Furthermore, with the configuration of the ECG main unit 100 and the electrode patch unit 200 as described above, the physiological monitoring apparatus 10 has a very compact structure and can be provided with a small form, for example, the ECG main unit 100 can have a 3-dimensional size as small as 37 mm*22.5 mm*7.7 mm, and have a weight as light as 7 grams; therefore, the physiological monitoring apparatus 10 may be imperceptible to the user while being worn, without influencing the normal activities of the user's daily living. That is, the wear experience of the ECG monitor apparatus 100 can also be improved.

In some case, the physiological monitoring apparatus 10 can be worn by a user for several days, while in other case, the physiological monitoring apparatus 10 can be worn for at least a week or for more than a week, for example ten days, fourteen days or even longer according to a necessary monitoring period as suggested by a doctor. After a physiological monitoring period is finished, the physiological monitoring apparatus 10 can be removed from the human body; in this circumstance, the ECG main unit 100 can be easily disassembled from the electrode patch unit 200 in a reverse process to the assemble process as shown in FIG. 12. Since the electrode patch unit 200 is disposable and low cost, the used electrode patch unit 200 can be discarded; when the user needs to take new physiological monitoring period, he or she only needs to change to a new electrode patch unit 200, and then assemble the ECG main unit 100 to the new electrode patch unit 200 to obtain a new ECG monitoring apparatus, which can be affixed to the human body of the user for a new monitoring period. As can be seen, the ECG monitor apparatus 100 as provided according to the embodiment of the present application is low cost for the user.

As a further improvement of the present application, a charge dock 300 as illustrated in FIG. 13, is further provided, which can be used for charging the battery 130 of the ECG main unit 100 and providing data transmission interface for the ECG main unit 100, so that the physiological signals collected by the ECG main unit 100 can be further be transmitted to the host with a cable 350, such as a USB-C cable.

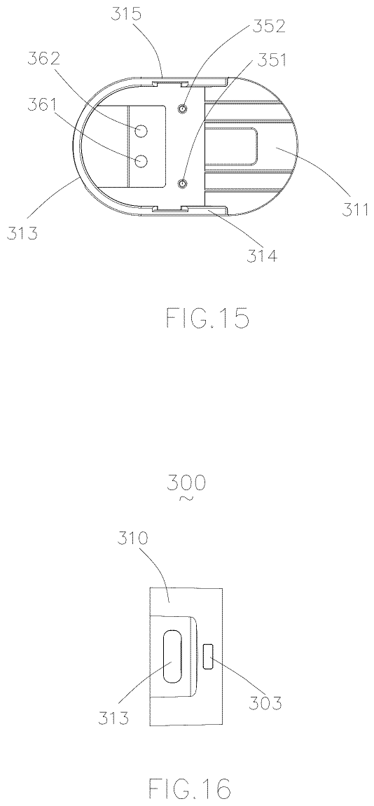

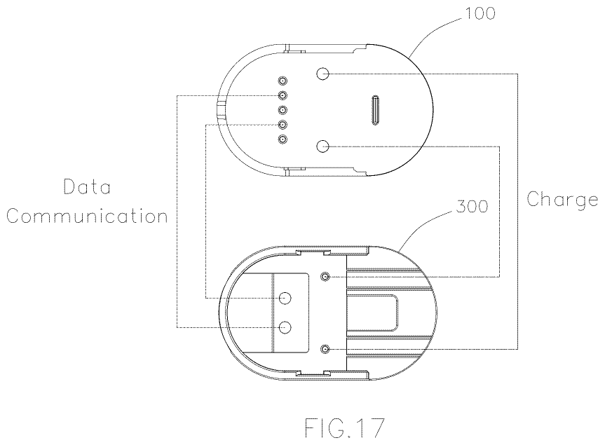

Referring to FIGS. 13-16, the charge dock 300 according to an embodiment of the present application includes a dock housing 310, a rear cover 320, and a circuit board module 330.

The dock housing 310 is configured for being engaging with and electrically coupled to the ECG main unit 100, which includes a main body 311 and a semi-enclosure wall 312 extending upwards from a peripheral margin of the main body 311. The rear cover 320 can be fixed to a bottom of the main body 311 to form a receiving space, and the circuit board module 330 can be received in the receiving space. The dock housing 310 and the rear cover 320 may also be made of engineering plastics such as Polycarbonate and Acrylonitrile Butadiene Styrene (PC+ABS).

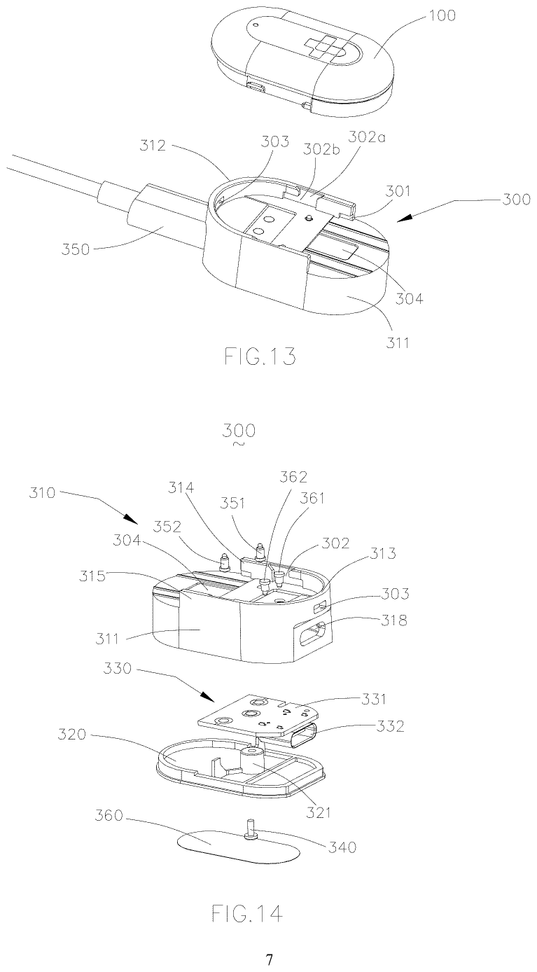

In an exemplary embodiment, the docking housing 310 may also be designed, as the bracket 210, for adaptively supporting and fixing the ECG main body 100, thus the semi-enclosure wall 312 of the docking housing 310 may have a configuration similar to or substantially same as that of the semi-enclosure wall 212 of the bracket 210. For example, as shown in FIG. 14, the semi-enclosure wall 312 may also include an arc-wall 313 corresponding to the connecting wall 191 of the rear case 120, and a pair of plane-walls 314 and 315 parallel to each other and corresponding to the plane-walls 193 and 194 of the rear case 120 respectively.

The arc-wall 313 may includes a connecting hole 303 formed on a main central portion thereof, which is also configured for receiving the connecting protrusion 103 of the ECG main unit 100; and two ends of the arc-wall 313 are connected to the pair of plane-walls 314 and 315 of the docking housing 310 respectively. Each of the plane-walls 314 and 315 also includes a sliding groove 302 and a receiving notch 301 formed on a bottom portion of an inner surface thereof. The receiving notch 301 is arranged at a distal end of a corresponding one of the plane-walls 314 and 315, which is adapted for receiving and limiting a position of the stopper block 101 of the rear case 120. The sliding groove 302 is arranged adjacent to the arc-wall 313, and includes a guiding portion 302a and a sliding portion 302b. The guiding portion 302a and the sliding portion 302b are communicated with each other to form an L-shaped groove. The guiding portion 302a is formed above the sliding portion 302b, and is configured for guiding a corresponding sliding block 102 of the first plane-wall 193 or 194 to move into the sliding portion 302b during assembly. The sliding portion 302b is adapted for enabling the corresponding sliding block 102 to slide therein towards the arc-wall 313.

Moreover, an upper surface of the main body 311 of the dock housing 310 may also include a latch member 304. The latch member 304 has a configuration substantially same as the latch member 204 of the bracket 210, which is arrange at a region corresponding to the locking protrusion 104 of the rear case 120, and is adapted for latching on the locking protrusion 104 when the ECG main body 100 is assembled to the charge dock 300.

In addition, the charge dock 300 further includes a pair of power pins 351 and 352, and a pair of transmission pins 361 and 362. The pair of power pins 351 and 352 are configured for electrically contacting the pair of power pins 151 and 152 when the ECG main unit 100 is assembled to the charge dock 300, so that a charging current can be transmitted from the circuit board module 330 to the ECG main unit 100 via the pair of power pins 351 and 352 as well as the pair of charging pins 151 and 152, to charge the battery 130 of the ECG main unit 100.

The pair of transmission pins 361 and 362 are configured for electrically contacting a pair of data pins selected from the plurality of data pins 161 to 165; for example, in the illustrated embodiment, a first data pin 162 and a second data pin 164 of the ECG main unit 100. The pair of transmission pins 361 and 362 are used, cooperatively with the pair of data pins 162 and 164, for performing data transmission between the ECG main unit 100 and the circuit board module 330 of the charge dock 300.

In other words, the first data pin 162 and the second data pin 164 of the ECG main unit 100 are enabled with two functions in different operation modes, one is for collecting the physiological signals from the electrode patch unit 200 when the ECG main unit 100 is assembled to the electrode patch unit 200 and affixed to the human body, and the other one is for transmitting the physiological signals after being processed from the ECG main unit 100 to the circuit board module 330 of the charge dock 300 when the ECG main unit 100 is assembled to charge dock 300.

The pair of power pins 351 and 352 and the pair of transmission pins 361 and 362 can be installed within the dock housing 310 in a reverse manner. For example, the pai of power pins 351 and 352 may be fixed, for example, in an upright structure, each of the power pins 351 and 352 has an elastic probe penetrating out of the upper surface of the main body 311 to electrically contact the first data pin 162 and a second data pin 164 of the ECG main unit 100, and a barrel contact end being exposed on a bottom of the dock housing 310 to electrically contact a corresponding charging pad on the circuit board module 330. The pair of transmission pins 361 and 362 may be conversely fixed, for example, in a downright structure, each of the transmission pins 361 and 362 includes an elastic plunger or probe penetrating out of a bottom surface of the main body 311 to electrically contact a corresponding data transmission pads on the circuit board module 330, and a barrel contact end being exposed on a bottom of the dock housing 310 to electrically contact a corresponding data pin 162 or 164 of the ECG main unit 100.

The circuit board module 330 may be a charge printed circuit board assembly (PCBA), which include a printed circuit board 331, a cable connector 332 arranged at a lower surface of the printed circuit board 331, a pair of charging pads (not labeled) and a pair of transmission pads (not labeled) formed on an upper surface of the printed circuit board 331. The pair of charging pads are configured for electrically contacting a pair of barrel contact ends of the power pins 351 and 352, and the pair of transmission pads are configured for electrically contacting the elastic probes of the pair of transmission pins 361 and 362. The cable connector 332 may be a USB-C connector for connecting to a USB-C cable 350, which is further connected to a host. By use of the cable connector 332 and the USB-C cable 350, the physiological signals including the ECG data collected and processed by the ECG main body 100 can be transmitted to the host via the charge dock 300.

In addition, the circuit board module 330 can be fixed to the rear cover 320 via a screw bolt 340, for example, a fixing post 321 having a through hole can be arranged at a main central region of the rear cover 320, the circuit board module 330 may further include a threaded hole 334 which is aligned with the through hole of the fixing post 321. The screw bolt 340 can pass through the fixing post 321 from a bottom of the rear cover 320, and is further threadedly fixed to the circuit board module 330 through the threaded hole 334. Furthermore, an opening 318 can be formed at the dock housing 310, which provides an access for the USB-C cable 350 to plug in the cable connector 332, so as to charge the battery 130 of the ECG main unit 100 via the charge duck 130, as well as to transmit the physiological signals from the ECG main unit 100 to the host.

In an optional embodiment, the charge dock 300 may further include a protection label 360 attached to a bottom to the rear cover 320 for protecting the charge dock 300 and provide some instruction of use of commercial introduction of the charge dock 300.

With these configurations, after the ECG main unit 100 is dissembled from the electrode patch unit 200, the ECG main unit 100 can further be installed to the charge dock 300. Referring also to FIG. 14, in this circumstance, the pair of the changing pins 151 and 152 in the main unit 100 can be electrically coupled to the pair of the power pins 351 and 352 in the charge dock 300 for charging, while the pair of the data pins 162 and 164 in the ECG main unit 100 can be electrically coupled to the pair of the transmission pins 361 and 362 in the chare dock 300 for data transmission. Therefore, the ECG main unit 100 can performed electrical charging and data transmission via the charge dock 300, this is more applicable to hospital scenario, in which the hospital can obtain physiological signals of several ECG main units 100 from different patients simultaneously, and can therefore improve diagnostic efficiency.

Although the above-described embodiments take the ECG main unit 100 for monitoring ECG data and other heart signals as an example, it should be noted, however, that, the concept of the present application can also be applicable to other physiological signal monitoring, for example, continuous glucose monitoring (CGM), dynamic blood pressure monitoring (BPM) or blood oxygen detection, etc. As such, in other alternative embodiments, the ECG main unit 100 can be alternatively replaced by a CGM main unit, a BPM main unit or other physiological main unit, which can be obtained the same improvement and technical effect as the illustrated embodiments.

Similarly, while operations are depicted in the drawings in a particular order, this should not be understood as requiring that such operations be performed in the particular order show or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. An apparatus for monitoring physiological signals comprising:
a physiological main unit comprising a sensor assembly;
an electrode patch unit being detachably engaged to the physiological main unit, the electrode patch unit comprising a plurality of conductive electrodes being affixed to a human body to detect physiological signals from the human body,
wherein the sensor assembly is electrically coupled to the conductive electrodes of the electrode patch unit and configured for collecting the physiological signals from the electrode patch unit.

2. The apparatus of claim 1, wherein the physiological main unit is an electrocardiogram (ECG) main unit comprising an ECG sensor assembly, and the physiological signals comprise heart signal including ECG data.

3. The apparatus of claim 1, wherein the physiological main unit further comprises a top cover, and a rear case fixed to the top cover, wherein the sensor assembly is received between the top cover and the rear case, and the rear case is detachably engaged to the electrode patch unit.

4. The apparatus of claim 3, wherein the electrode patch unit comprises:
a socket for being engaged to the rear case of the physiological main unit;
a line film arranged under the socket and comprising a plurality of conductive traces for being electrically coupled to the sensor assembly of the physiological main unit; and
wherein the plurality of conductive electrodes are electrode gel members attached to the line film via a medical tape and electrically coupled to the conductive traces of the line film.

5. The apparatus of claim 4, wherein the rear case of the physiological main unit comprises a pair of first plane-walls opposite to each other and each comprising a sliding block and a stopper block;
wherein the socket comprises a plurality of second plane-walls corresponding to the pair of first plane-walls of the rear case, each of the second plane-walls comprises a first sliding groove and a first receiving notch formed at a bottom portion of an inner surface of the plane-wall;
wherein the sliding block of the rear case is configured for sliding along the first sliding groove when the physiological main unit is engaged to the electrode patch unit, and the first receiving notch is configured for receiving the stopper block and limiting a position of the stopper block after the physiological main unit being engaged to the electrode patch unit.

6. The apparatus of claim 5, wherein the first sliding groove comprises a guiding portion and a sliding portion communicated with each other to formed an L-shaped groove,
wherein the guiding portion is formed above the sliding portion, and is configured for guiding a corresponding sliding block to move into the sliding portion, the sliding portion is adapted for enabling the corresponding sliding block to slide therein.

7. The apparatus of claim 5, wherein the rear case further comprises a connecting wall and a free wall opposite to each other and being connected between the par of the first plane-wall respectively to form an enclosure wall structure, wherein the connecting wall and the free wall are both in an arc-wall form, and a radius of the free wall is greater than that of the connecting wall.

8. The apparatus of claim 7, wherein the connecting wall comprises a connecting protrusion being arranged at an outer middle portion thereof;
the bracket further comprises an arc-wall corresponding to the connecting wall and comprising a connecting groove, the connecting groove is configured for receiving the connecting protrusion when the physiological main unit is engaged to the electrode patch unit.

9. The apparatus of claim 7, wherein the physiological main unit further comprises a plurality of data pins, each of the data pins is a pogo pin, and a bottom plate of the rear case comprises a plurality of mounting structure for fixing the data pins respectively;
wherein an elastic probe of each of the data pins penetrates out of a bottom of the rear case for being electrically coupled to the conductive traces in the electrode patch unit, and a barrel contact end of each of the data pins is exposed on an upper surface of the bottom plate for being electrically coupled to the sensor assembly.

10. The apparatus of claim 9, wherein the physiological main unit further comprises a pair of charging pins, each of the charging pins is also a pogo pin, and the rear case further comprises a pair of mounting posts arranged at the bottom plate for fixing the charge pins respectively;
wherein an elastic probe of the charging pin penetrates out of a top of the mounting post for electrically contacting a corresponding pad on the sensor assembly, and a barrel contact end of the charging pin is exposed on the bottom of the rear case for electrically coupled to a charge dock for receiving a charging current.

11. The apparatus of claim 1, wherein the electrode patch unit is a disposable element which is detached from the physiological main unit after use, such that a new electrode patch unit is capable of being engaged to the physiological main unit for a new physiological monitoring period.

12. The apparatus of claim 10, further comprising a charge dock for charging a battery of the physiological main unit and providing data transmission interface for the physiological main unit, so as to enable the physiological signals collected by the physiological main unit to be transmitted to a host.

13. The apparatus of claim 12. wherein the charge dock comprises a dock housing, a rear cover and a circuit board module; the rear cover is fixed to a bottom of the dock housing to form a receiving space for receiving the circuit board module, the circuit board module comprises a printed circuit board and a cable connector arranged at the printed circuit board and for being connected to the host via a cable.

14. The apparatus of claim 13, wherein the dock housing is designed for adaptively supporting and fixing the physiological main unit when the physiological main unit needs charging or data transmission and is detached from the electrode patch unit; the dock housing comprises a main body and a semi-enclosure wall extending upwards from a peripheral margin of the main body.

15. The apparatus of claim 14, wherein the semi-enclosure wall of the dock housing comprises a pair of third plane-walls opposite to each other and corresponding to the pair of first plane-walls of the rear case, and an arc-wall being connected between the pair of third plane-walls and corresponding to the connecting wall of the rear case.

16. The apparatus of claim 15, wherein the arc-wall of the dock housing comprises a connecting hole formed on a main central portion thereof and for receiving the connecting protrusion of the physiological main unit; each of the third plane-walls comprises a second sliding groove and a second receiving notch formed on a bottom portion of an inner surface thereof, the second sliding groove and the second receiving notch has a configuration substantially same as that of the first sliding groove and the first receiving notch of the bracket of the electrode patch unit.

17. The apparatus of claim 15, wherein the charge dock further comprises
a pair of power pins configured for electrically contacting the pair of charging pins of the physiological main unit when the physiological main unit is engaged to the charge dock; and
a pair of transmission pins configured for electrically contacting a pair of data pins of the physiological main unit; wherein the pair of transmission pins are used cooperatively with the selected pair of data pins for performing data transmission between the physiological main unit and the charge dock.

18. A charge dock for providing charging and data transmission for a physiological main unit, the physiological main unit comprising a sensor assembly for collecting physiological signals from a human body, the charge dock comprising:
a dock housing being detachably engaged to the physiological main unit;
a rear cover being fixed to the dock housing to form a receiving space; and
a circuit board module received in the receiving space and being electrically connected to the physiological main unit,
wherein the circuit board module comprises a printed circuit board and a cable connector arranged at the printed circuit board, the cable connector is configured for being connected to a cable for providing a charge current to the physiological main unit and transmitting the physiological signals collected by the physiological main unit to a host.

19. The charge dock of claim 18, wherein the dock housing comprises a main body and a semi-enclosure wall extending upwards from a peripheral margin of the main body;
wherein the semi-enclosure wall of the dock housing comprises a pair of plane-walls opposite to each other, and an arc-wall being connected between the pair of plane-walls;
wherein the arc-wall of the dock housing comprises a connecting hole formed on a main central portion thereof and for receiving a connecting protrusion of the physiological main unit;
each of the plane-walls comprises a sliding groove and a receiving notch formed on a bottom portion of an inner surface thereof, wherein a sliding block arranged on the physiological main unit is capable of sliding along the sliding groove when the physiological main unit is engaged to the charge dock, and the receiving notch is configured for receiving the stopper block and limiting a position of the stopper block after the physiological main unit being engaged to the electrode patch unit.

20. An electrocardiogram (ECG) monitoring apparatus, comprising
an ECG main unit comprising a top cover, and a rear case fixed to the top cove, and an ECG sensor assembly being received between the top cover and the rear case; and
an electrode patch unit being detachably engaged to the ECG main unit, the electrode patch unit comprising a socket for being engaged to the rear case of the physiological main unit, a plurality of conductive electrodes being affixed to a human body to detect heart signal including an ECG signal from the human body; a line film arranged under the socket and comprising a plurality of conductive traces for being electrically coupled to the ECG sensor assembly unit,
wherein the ECG sensor assembly is electrically coupled to the conductive electrodes of the electrode patch unit and configured for collecting the physiological signals from the electrode patch unit.
